SEQUENCE LISTING (28 sequences)

SEQ ID NO: 1 - protein, 17 aa, Artificial Sequence
  Cell killing fusion peptide
CNGRCGGKLLNLISKLF

SEQ ID NO: 2 - protein, 17 aa, Artificial Sequence
  Cell killing fusion peptide
CGKRKGGKLLNLISKLF

SEQ ID NO: 3 - protein, 17 aa, Artificial Sequence
  Cell killing fusion peptide
KLLNLISKLFGGCGKRK

SEQ ID NO: 4 - protein, 18 aa, Artificial Sequence
  Cell killing fusion peptide
WIFPWIQLKLLNLISKLF

SEQ ID NO: 5 - protein, 19 aa, Artificial Sequence
  Cell killing fusion peptide
RLLRLLRGGKLLNLISKLF

SEQ ID NO: 6 - protein, 19 aa, Artificial Sequence
  Cell killing fusion peptide
KLLNLISKLFGGRLLRLLR

SEQ ID NO: 7 - protein, 19 aa, Artificial Sequence
  Cell killing fusion peptide
RPARPARGGKLLNLISKLF

SEQ ID NO: 8 - protein, 19 aa, Artificial Sequence
  Cell killing fusion peptide
KLLNLISKLFGGRPARPAR

SEQ ID NO: 9 - protein, 19 aa, Artificial Sequence
  Cell killing fusion peptide
RGDRGDLGGKLLNLISKLF

SEQ ID NO: 10 - protein, 19 aa, Artificial Sequence
  Cell killing fusion peptide
KLLNLISKLFGGLGDRGDR

SEQ ID NO: 11 - protein, 22 aa, Artificial Sequence
  Cell killing fusion peptide
WDLAWMFRLPVGKLLNLISKLF

SEQ ID NO: 12 - protein, 19 aa, Artificial Sequence
  Cell killing fusion peptide
CGRDKGPDCKLLNLISKLF

SEQ ID NO: 13 - protein, 19 aa, Artificial Sequence
  Cell killing fusion peptide
KLLNLISKLFCGRDKGPDC

SEQ ID NO: 14 - protein, 20 aa, Artificial Sequence
  Cell killing fusion peptide
KLLNLISKLFCGRDKRLYDC

SEQ ID NO: 15 - protein, 19 aa, Artificial Sequence
  Cell killing fusion peptide
CRGDKGPDCKLLNLISKLF

SEQ ID NO: 16 - protein, 19 aa, Artificial Sequence
  Cell killing fusion peptide
KLLNLISKLFCRGDKGPDC

SEQ ID NO: 17 - protein, 20 aa, Artificial Sequence
  Cell killing fusion peptide
KLLNLISKLFCRGDKRLYDC

SEQ ID NO: 18 - protein, 17 aa, Artificial Sequence
  Cell killing fusion peptide
CRGDKGGKLLNLISKLF

SEQ ID NO: 19 - protein, 17 aa, Artificial Sequence
  Cell killing fusion peptide
KLLNLISKLFGGCRGDK

SEQ ID NO: 20 - protein, 7 aa, Artificial Sequence
  cancer targeting domain
RPARPAR

SEQ ID NO: 21 - protein, 10 aa, Artificial Sequence
  Cell Killing Peptide
KLLNLISKLF

SEQ ID NO: 22 - protein, 10 aa, Artificial Sequence
  Cell Killing Peptide
KALNLISKLF

SEQ ID NO: 23 - protein, 10 aa, Artificial Sequence
  Cell Killing Peptide
KLAALISKLF

SEQ ID NO: 24 - protein, 10 aa, Artificial Sequence
  Cell Killing Peptide
KLLNLIAALF

SEQ ID NO: 25 - protein, 10 aa, Artificial Sequence
  Cell Killing Peptide
KALNLIAALF

SEQ ID NO: 26 - protein, 10 aa, Artificial Sequence
  Cell Killing Peptide
KAANLISKLF

SEQ ID NO: 27 - protein, 10 aa, Artificial Sequence
  Cell Killing Peptide
KLLNAISKLF

SEQ ID NO: 28 - protein, 10 aa, Artificial Sequence
  Cell Killing Peptide
KLLNLISKAF

(19) United States
(12) Patent Application Publication
(10) Pub. No.: US 2014/0343249 A1
(43) Pub. Date: Nov. 20, 2014

(54) CELL KILLING FUSION PEPTIDE EXHIBITING TUMOR CELL-SPECIFIC NECROSIS INDUCTION AND TUMOR REGRESSION

(71) Applicants: Tae-Hyoung Kim, Gwangju (KR); Ae Ran Moon, Damyang-gun (KR); Ji Young Kim, Gwangju (KR)

(72) Inventors: Tae-Hyoung Kim, Gwangju (KR); Ae Ran Moon, Damyang-gun (KR); Ji Young Kim, Gwangju (KR)

(73) Assignee: INDUSTRY-ACADEMIC COOPERATION FOUNDATION, CHOSUN UNIVERSITY, Gwangju (KR)

(21) Appl. No.: 14/361,891

(22) PCT Filed: Dec. 15, 2011
(86) PCT No.: PCT/KR2011/009678

§ 371 Date: Jul. 21, 2014

(30) Foreign Application Priority Data
Nov. 30, 2011 (KR) 10-2011-0127120

(51) Int. Cl. C07K 7/08; C07K 16/18

(52) U.S. Cl. CPC C07K 7/08; C07K 16/18; C07K 2319/07; C07K 2319/33
USPC 530/326; 536/23.4; 435/320.1; 435/69.7; 530/387.9; 435/252.3

(57) ABSTRACT

A cell-killing peptide, more specifically a cell-killing CKP fusion peptide (CTD7:CKP) is disclosed, wherein a cell-killing peptide (CKP) comprising 10 amino acids in MTD of Noxa protein causing cell death, and 7 amino acids targeting a cancer cell are fused. The cell-killing CKP fusion peptide induces strong cell necrosis at various cancer cell lines (HeLa, HCT116, MCF-7, A549, BJAB, CT26, PC3 and the like) and shows strong tumor regression effect at a mouse tumor model using experimental animals, but does not show apoptosis at normal cells. Therefore, it can be broadly used to human body for treating various diseases requiring cell death, particularly, as an anti-cancer drug.

### TECHNICAL FIELD

The present invention relates to a cancer targeting cell-killing fusion peptide, more specifically to a cell-killing CKP fusion peptide (CTD7:CKP) showing cancer cell necrosis effect and strong tumor regression effect at a mouse tumor model using experimental animals, which can be broadly used for treating various diseases requiring cell death due to excessive cell proliferation, particularly, as an anti-cancer drug.

### BACKGROUND ART

Cell necrosis is irreversible cell death caused by excessive cell toxicity and continuous environmental stress, and unlike apoptosis, it does not use cellular energy. The present inventors firstly found a mitochondria targeting domain (MTD) at the C-terminal region from human Noxa, other than a BH3 domain, which plays an important role for inducing apoptosis (Seo et. al., 2003, JBC, 278, 48292-48299).

The MTD is a part of the Noxa protein, which is well known to cause apoptosis, and it is known that the MTD helps movement of the Noxa to mitochondria and apoptosis is caused by the BH3 domain of the Noxa. In the present invention, this is named cell-killing peptide (CKP). The MTD of the Noxa has strong cell necrosis effect, which can kill 80% or more cells in 10 minutes by increasing calcium concentration in cytosol, and the calcium concentration increase in cytosol is caused by calcium release from mitochondria in cells, not by calcium release from endoplasmic reticulum (ER) or calcium influx from outside, which is known as a general cause of cell necrosis. It shows that excessive calcium can be actively released at a time from mitochondria whose calcium amount is less than that of in ER, and it may be enough to cause cell necrosis (Seo et. al., 2009, Cancer Res, 69 (21):8356-8365).

Physiological examples of cell necrosis may be cell death by ischemia: reperfusion, excitotoxicity of nerve cells in ischemic stroke and anti-cancer drugs (e.g., photodynamic cancer-therapy drugs). In recent, according to the result from cell death study, there may be some vague cases that the criteria of the cell death are clearly arranged. In the case of ischemia: reperfusion, cells in ischemia state produce ATP by glycolysis, and when oxygen is suddenly supplied, those produce ATP by oxidative phosphorylation. It is known that in this process, mitochondria produces a lot of ROS, and thereby cell necrosis occurs. In the case of N-methyl-D-aspartic acid (NMDA)-mediated excitotoxicity of a cortical neuron used as an in vitro model of ischemic stroke, it is known that when excessive neurotransmission is continued to a neuron, intracellular calcium concentration increases, and necrosis is induced by calcium. Further, PS flip-flopping phenomenon of exposing phosphatidylserine (PS) out of the cell membrane is observed together with pyknotic nuclei observed in necrosis (Wang et. al, 2004).

In recent, cell necrosis-inducing materials are being developed as therapeutic agents for diseases caused by excessive cell proliferation. As the synthetic peptide showing anti-cancer effect by inducing cell necrosis, it was currently reported that Kaisin, Kaisinl and Kaisinll, buforin derivatives, selectively act on only cancer cells, and therefore, they can be usefully used as an active ingredient of effective anti-cancer drugs (Korean Patent Application No.:10-2007-0001591). LTX-315 named lytic peptide developed by Lytix Biopharma, Norwegian bio pharmaceutical company, is currently on the clinical phase I and II study, and it was reported that the peptide has characteristics of inducing of very rapid tumor lysis and cell necrosis. Further, in Korea, clinical trial of JX-594, which is being developed by Green Cross Corp. and Jennerex (USA), conducted on liver cancer patients, and according to the result of the clinical trial by co-administration of JX-594 and liver cancer drug, sorafenib, brilliant cancer cell necrosis inducing effect was observed.

The present inventors completed the present invention about a novel cell-killing peptide (CKP) by finding that the MTD domain, which exists in c-terminus of the existing Noxa protein and is known as just playing a role in mitochondria targeting by assisting BH3 domain, can strongly cause cell death of cancer cell lines (for example, HeLa, HCT116) when it is combined with R8, protein transduction domain (PTD) (Korean Patent Registration No:10-685345).

In vivo phage display is the optimized method for searching small peptide ligands targeting specific cells and tissues in a short time, and this tissue-specific peptide search combined with drug delivery system is useful to delivery system of protein drugs, which are difficult to be delivered into the body. Rouslahti et al selected cancer cell targeting peptide, which targets specific tumor tissue, by using T7 phage display in order to inhibit proliferation of vessels required for tumor growth as well as proliferation of tumor.

In this background, the present inventors completed the present invention by identifying strong cell-killing activity in cancer cell lines (for example, HeLa, HCT116, MCF-7 and the like) and strong tumor regression effect and cytotoxicity in a mouse tumor model using experimental animals of cell-killing CKP fusion peptides, wherein cell-killing peptide (CKP) comprising 10 amino acids in the MTD region of the Noxa protein caused cell death and 7 amino acids targeting cancer cell are fused.

### DISCLOSURE

### Technical Problem

Accordingly, the present invention is objected to provide a cell-killing CKP fusion peptide (CTD7:CKP), wherein a cell-killing peptide (CKP) comprising 10 amino acids in MTD of Noxa protein causing cell death, and 7 amino acids targeting a cancer cell (CTD7) are fused.

Further, the present invention is objected to provide a pharmaceutical composition comprising the fusion peptide, which shows cell-killing effect and tumor regression effect.

### Technical Solution

Herein, the term “peptide” refers to a linear molecule, which is formed by linking amino acid residues one another through peptide bonds.

The peptide of the present invention may be manufactured by chemical synthetic methods known in the art, particularly, solid-phase synthesis techniques (Merrifield, J. Amer. Chem. Soc. 85:2149-54 (1963); Stewart, et al., Solid Phase Peptide Synthesis, 2nd. ed., Pierce Chem. Co.: Rockford, 111 (1984)).

The cell-killing peptide (CKP) of the present invention is named cell-killing peptide (CKP) after the present inventors firstly found that a peptide, which has been known as a mitochondria targeting domain in Noxa causing apoptosis, has an activity to effectively kill cancer cells. The cell-killing peptide (CKP) contains the amino acid sequence of SEQ ID NO: 21 (KLLNLISKLF).

According to one embodiment of the present invention, the present invention provides a cancer targeting cell-killing fusion peptide, which comprises the following cancer cell targeting domain (CTD; R₁-R₇) and cell-killing peptide (CKP), and a linker sequence between thereof, and arranged with CTD (R₁-R₇)-Linker-CKP or CKP-Linker-CTD (R₁-R₇) configuration:

(a) the cancer cell targeting domain (CTD; R₁-R₇) consisting of an amino acid sequence expressed as Arg-Xaa-Xaa-Arg-Xaa-Xaa-Arg;

(b) the cell-killing peptide (CKP) consisting of the amino acid sequence of SEQ ID NO: 21 (KLLNLISKLF) or its homologous sequences having at least 70% of sequence homology; and

(c) the linker having an amino acid sequence, wherein n=0-5.

In the present invention, the cancer cell targeting domain (CTD; R₁-R₇) may have any amino acid sequence expressed as Arg-Xaa-Xaa-Arg-Xaa-Xaa-Arg. The present inventors firstly found that the sequence having common rule, wherein the 1^{st}, 4^{th} and 7^{th} amino acids are arginine (Arg), is effective to cancer cell targeting. Preferably, the present invention provides the cancer targeting cell-killing fusion peptide, which is characterized that the cancer cell targeting domain (CTD; R₁₋R₇) may consist of the amino acid sequence of SEQ ID NO: 20 (RPARPAR).

In the present invention, the cell-killing peptide (CKP) may show cell-killing activity when it has at least 70% of sequence homology with the amino acid sequence of SEQ ID NO: 21. In Examples of the present invention, it is demonstrated that a variant, wherein 1, 2 or 3 amino acid sequences of the 10 amino acid sequences of the CKP are substituted, also has cell-killing activity. Specifically, the cell-killing peptide (CKP) having at least 70% sequence homology with the amino acid sequence of SEQ ID NO: 21 is characterized by consisting of the amino acid sequence of SEQ ID NO: 22 (KALNLISKLF), SEQ ID NO: 23 (KLAALISKLF), SEQ ID NO: 24 (KLLNLIAALF) or SEQ ID NO: 25 (KALNLIAALF), and more preferably, the cell-killing peptide (CKP) having at least 70% sequence homology with the amino acid sequence of SEQ ID NO: 21 is characterized that the 2^{nd}, 3^{rd}, 5^{th} and 9^{th} amino acid sequence of SEQ ID NO: 21 are leucines. In the cell-killing peptide of the amino acid sequence of SEQ ID NO: 21, leucines are repeatedly appeared at the 2^{nd}, 3^{rd}, 5^{th} and 9^{th} sequences, and it is demonstrated that the sequences play important role in cell death capacity by experiments in Examples.

In the present invention, the cancer cell targeting domain (CTD; R₁-R₇) and the cell-killing peptide (CKP) may be linked each other without the linker sequence, but preferably, those may be linked each other through a linker, which has an amino acid sequence with enough length not to interfere with the cell-killing activity of the CKP (n=0-5; n is the number of amino acid sequence). The linker may be any linker known in the art (see Huston, et al., Methods in Enzymology, 203:46-88 (1991); and Whitlow, et al., Protein Eng., 6:989 (1993)). Specifically, the linker, which is suitable for the present invention, may mainly consist of glycine or serine amino acid, and other amino acids, and its length may be 2-5 amino acids. In Examples of the present invention, a linker consisting of 2 Gly residues was used.

According to another embodiment of the present invention, the present invention provides a DNA or RNA oligonucleotide encoding the cancer targeting cell-killing fusion peptide according to the present invention. The DNA or RNA oligonucleotide may be manufactured by chemical synthesis based on codons encoding the cancer targeting cell-killing fusion peptide in an automatic synthesizer. It may also be manufactured by PCR amplification or transcription of a synthesized gene.

According to another embodiment of the present invention, the present invention provides a recombinant vector comprising the DNA oligonucleotide according to the present invention, and a cell transformed with the recombinant vector. The recombinant vector may be manufactured by inserting the DNA oligonucleotide into a plasmid or a virus vector according to the methods known in the art. The methods used for manufacturing the vector is broadly known to those skilled in the art and are described in various published documents. Particularly, techniques for manufacturing the suitable vector comprising functional and regulatory component, for example, promoter, enhancer, termination and polyadenylation signals, selection marker, replication origin and splicing signal, are known to those skilled in the art. A eukaryotic expression vector may contain prokaryotic sequences generally promoting vector amplification in bacteria, for example, replication origin, and antibiotics resistant gene for selection in bacteria. Various eukaryotic expression vectors containing a cloning site, to which a polynucleotide can be operably linked, are broadly known to those skilled in the art, and some of them are commercially available from companies (e.g.: Stratagene, La Jolla, Calif.; Invitrogen, Carlsbad, Calif.; Promega, Madison, Wis.; or BD Biosciences Clontech, Palo Alto, Calif.).

According to another embodiment of the present invention, the present invention provides a method for manufacturing the cancer targeting cell-killing fusion peptide comprising: culturing the transformed cell according to the present invention and isolating the cancer targeting cell-killing fusion peptide from the cultured cell.

According to another embodiment of the present invention, the present invention provides a method for manufacturing the cancer targeting cell-killing fusion peptide by chemical synthesis according to solid phase peptide synthesis comprising: sequentially linking amino acids arranged with the CTD (R₁-R₇)-Linker-CKP or CKP-Linker-CTD (R₁-R₇) configuration according to the present invention to a polymer scaffold, followed by finally separating thereof from the polymer scaffold.

According to another embodiment of the present invention, the present invention provides an antibody produced by using the cancer targeting cell-killing fusion peptide according to the present invention as an antigen.

A polyclonal antibody may be manufactured by injecting the cancer targeting cell-killing fusion peptide as an immunogen to an external host according to conventional methods known to those skilled in the art. The external host may be mammals such as mouse, rat, sheep and rabbit. The immunogen may be injected by intramuscular, intraabdominal or subcutaneous injection, and it may be generally injected with an adjuvant for improving antigenicity. Serum showing improved titer and specificity to an antigen may be harvested by periodically collecting blood from the external host, or an antibody is isolated and purified therefrom. A monoclonal antibody may be manufactured according to immortalized cell line producing technique by fusion, known to those skilled in the art (Koeher and Milstein (1975) Nature, 256:495). As briefly explaining the method, firstly, the cancer targeting cell-killing fusion peptide is synthesized, combined with bovine serum albumin, and immunized to a mouse. Then, antigen-producing lymphocyte isolated from the mouse is fused with human or mouse myeloma to produce an immortalized hybridoma, and then the hybridoma cells producing the desired monoclonal antibody are selected by ELISA followed by isolating and purifying the monoclonal antibody from culture.

According to another embodiment, the present invention provides a PEG variant of the cancer targeting cell-killing fusion peptide, which is characterized that PEG is linked to the cancer targeting cell-killing fusion peptide according to the present invention. The PEG variant may be manufactured by linking polyethylene glycol (PEG) to the cancer targeting cell-killing fusion peptide according to PEGylation method known before. For example, for improving stability during blood circulation and reducing immunogenicity, mPEG aldehyde may be subjected to solid-phase PEGylation to a-amine group at N-terminus of the cancer targeting cell-killing fusion peptide.

As the most preferable embodiment, the cell-killing CKP fusion peptide (CTD7:CKP) of the present invention contains the amino acid sequence of the cancer cell targeting peptide, Arg-Pro-Ala-Arg-Pro-Ala-Arg (RPARPAR), and the amino acid sequence of the cell-killing peptide, Lys-Leu-Leu-Asn-Leu-Ile-Ser-Lys-Leu-Phe (KLLNLISKLF) (FIG. 1). The cell-killing CKP fusion peptide (CTD7:CKP) of the present invention, wherein the cell-killing peptide and the cancer cell targeting peptide are fused, may be chemically synthesized according to solid phase peptide synthesis, which may manufacture a peptide with high purity by sequentially linking amino acids to a polymer scaffold followed by isolated from the polymer scaffold finally. Further, it may be manufactured by synthesizing the oligonucleotide encoding the peptide at an automatic synthesizer, or the base sequences coding the MTD domain is selectively amplified from base sequences of Noxa gene (NCBI GenBank number: NM_{—}021127) by polymerase chain reaction (PCR) followed by inserted into a proper vector, expressed through in vivo transcription and translation, and then purified.

The pharmaceutical synthetic peptide of the present invention is characterized by being used for treating cancer cells. The pharmaceutical synthetic peptide of the present invention may be manufactured by common methods known in the pharmaceutical field, and it may be used as the fusion peptide itself or a formulation such as powder, granules, tablets, capsules and injection formulation by mixing with a pharmaceutically acceptable carrier, excipient, diluent and the like. Further, it may be administered parenterally. The dosage of the pharmaceutical composition of the present invention may be properly selected depending on factors such as age, sex and physical condition of a patient, severity of disease or symptom, administration period, administration method, discharge ratio, body weight, diet and so on. Preferably, the pharmaceutical composition of the present invention can be administered to an adult cancer patient in the range of 1-100 mg per day (active ingredient).

### Advantageous Effects

The present invention demonstrated that the cell-killing CKP peptide fused to the cancer cell targeting peptide can strongly induce cell death of cancer cell lines, and shows strong tumor regression effect in a mouse tumor model. It has not been known that this cell death occurs through which pathway, but it shows stronger cell death effect than general apoptosis.

### DESCRIPTION OF DRAWINGS

FIG. 1 is schematic diagrams of the cell-killing CKP fusion peptide (CTD7:CKP) of the present invention, wherein the cancer cell targeting domain (CTD) and the cell-killing peptide (CKP) are fused. A shows that the CTD domain at N-terminal is fused to the CKP domain at C-terminal. B shows that the CKP domain at N-terminal is fused to the CTD domain at C-terminal.

FIGS. 2A to 2C are graphs showing cell necrosis inducing activity of various cell-killing CKP fusion peptides (CTDI :CKP to CTD12:CKP) in a mouse colon cancer cell line, CT-26.

FIGS. 3A and 3B are graphs showing cell necrosis inducing activity of various cell-killing CKP fusion peptides (CTD13:CKP to CTD19:CKP) in a mouse colon cancer cell line, CT-26.

FIGS. 4A to 4G are graphs showing tumor regression effect of various cell-killing CKP fusion peptides in a mouse tumor model using experimental animals.

FIG. 5 is a graph showing increased cell necrosis inducing effect of the cell-killing CKP fusion peptide (CTD7:CKP) in a human cervical cancer cell line, HeLa and a human colon cancer cell line, HCT116.

FIG. 6 is a graph showing increased cell necrosis inducing effect of the cell-killing CKP fusion peptide (CTD7:CKP) in a human breast cancer cell line, MCF-7 and a human lung cancer cell line, A549.

FIG. 7 is a graph showing increased cell necrosis inducing effect of the cell-killing CKP fusion peptide (CTD7:CKP) in a human B-lymphoma cell line, BJAB and a mouse prostate cancer cell line, PC3.

FIG. 8 is a graph showing increased cell necrosis inducing effect of the cell-killing CKP fusion peptide (CTD7:CKP) in primary peritoneal macrophages and splenocytes as normal cells.

FIG. 9 is a schematic diagram of tumor regression stretagy of the cell-killing CKP fusion peptide (CTD7:CKP) in a mouse tumor model using experimental animals.

FIG. 10 is a graph showing tumor regression effect of the cell-killing CKP fusion peptide (CTD7:CKP) in a mouse tumor model.

FIG. 11 is a table showing the result of measuring the amounts of ALT and AST released to blood by the cell-killing CKP fusion peptide (CTD7:CKP) when the liver is damaged.

FIGS. 12A and 12B are pictures for histologically observing tumor regression effect of the cell-killing CKP fusion peptide by time.

FIG. 13 is pictures for histologically observing the liver for evaluating cytotoxicity of normal cells by the cell-killing CKP fusion peptide by time.

FIG. 14 is the result of cell-killing test by the variants of the CKP peptide.

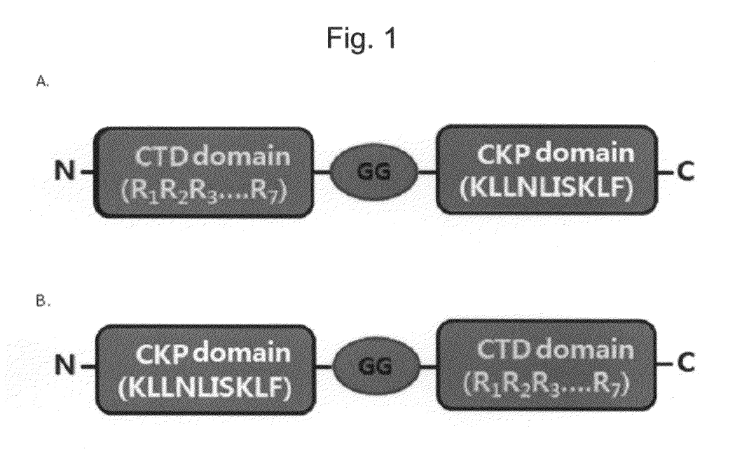

The patent or application file contains at least one drawing executed in color. Copies of this patent or patent application publication with color drawing(s) will be provided by the Office upon request and payment of the necessary fee.

### MODE FOR INVENTION

The examples and experiments will now be described. The following examples and experiments are for illustrative purposes only and not intended to limit the scope of the present disclosure.

1. Reagent

Reagents were purchased as follows:

HeLa, HCT116, A549, MCF-7, BJAB and PC3 as Human cancer cell lines, and CT-26 as a mouse cancer cell line were purchased from Korea Cell Line Bank; Dulbecco's Modified Eagle Medium, RPMI-1640 medium, Trypsin, Fetal bovine serum and Hematoxylin&Eosin dye were purchased from Sigma chemical co.; and XTT assay kit was purchased from Promega.

2. Animal

As Experimental animals, BALB/c mice of 6 weeks old (22-25 g) were used, and freely fed with feed (purina korea) and water. Breeding system was maintained at a temperature of 21-24° C. and a relative humidity of 40-80% in a 12-hour light/12-hour dark cycle.

### EXAMPLE 1

### Peptide Synthesis

The cell-killing CKP peptide (KLLNLISKLF) of the present invention was fused to a lots of cancer cell targeting peptides, which is expected to target at cancer tissues, in order to synthesize cell-killing CKP fusion peptides. In order to synthesize the peptide, manual Fmoc synthetic method using 0.25 mmol Unit was basically adopted. In detail, resin was washed by using 4× DMF, blended with 10 ml of 20% piperidine/DMF solution for 1 minute and separated to remove supernatant. After that, the resulting resin was mixed again with 10 ml of 20% piperidine/DMF solution, shaken for 30 min and then washed by using 4× DMF. Ninhydrin test was performed to identify whether the piperidine was remained or not (The resin appeared blue color when without piperidine). In order to proceed a coupling step, a solution comprising 1 mmol of Fmoc-amino acid, 2.1 ml of 0.45 M HBTU/HOBT (1 mmol) and 348 μl of DIEA (2 mmol) was prepared. The resin was blended with the solution, stirred for 30 min, poured out to discard the solution and then washed by using 4× DMF. In order to perform coupling of amino acids, the coupling step mentioned above was repeated and as a result, many kinds of cancer cell targeting peptides of the present invention combined with the cell-killing peptide were synthesized. The resulting peptides prepared through the above procedure were characterized by using HPLC (Instrument: Waters 2690 separations module, Flow rate: 1.0 ml/min, Gradient: 0%-20% B 5 minutes 20%-50% B 20 minutes 50%-80% B 5 minutes, A; 0.1% TFA water, B; 0.1% TFA acetonitrile, column: Waters C18, 5 micron, Detection: 220 nm, purity: 95%) and mass spectroscopy (Instrument: HP 1100 series LC/MSD). The synthetic peptides were dissolved in water, adjusted to 1 mM of concentration and stored at −80° C.

TABLE 1
CTD:CKP Fusion Peptide SEQ ID NO and Amino Acid Sequence

| | SEQ | | | | |
|---|---|---|---|---|---|
| Kind of | ID | Sequence | Chain | Sequence | |
| CTD:CKP | NO | Form | Number | Type | Amino Acid Sequence |
| CTD1:CKP | 1 | Amino | single | Peptide | CNGRCGGKLLNLISKLF |
| | | Acid | | | |
| CTD2:CKP | 2 | Amino | Single | Peptide | CGKRKGGKLLNLISKLF |
| | | Acid | | | |
| CTD3:CKP | 3 | Amino | Single | Peptide | KLLNLISKLFGGCGKRK |
| | | Acid | | | |
| CTD4:CKP | 4 | Amino | Single | Peptide | WIFPWIQLKLLNLISKLF |
| | | Acid | | | |
| CTD5:CKP | 5 | Amino | Single | Peptide | RLLRLLRGGKLLNLISKLF |
| | | Acid | | | |
| CTD6:CKP | 6 | Amino | Single | Peptide | KLLNLISKLFGGRLLRLLR |
| | | Acid | | | |
| CTD7:CKP | 7 | Amino | Single | Peptide | RPARPARGGKLLNLISKLF |
| | | Acid | | | |
| CTD8:CKP | 8 | Amino | Single | Peptide | KLLNLISKLFGGRPARPAR |
| | | Acid | | | |
| CTD9:CKP | 9 | Amino | Single | Peptide | RGDRGDLGGKLLNLISKLF |
| | | Acid | | | |
| CTD10:CKP | 10 | Amino | Single | Peptide | KLLNLISKLFGGLGDRGDR |
| | | Acid | | | |
| CTD11:CKP | 11 | Amino | Single | Peptide | WDLAWMFRLPVGKLLNLISKLF |
| | | Acid | | | |
| CTD12:CKP | 12 | Amino | Single | Peptide | CGRDKGPDCKLLNLISKLF |
| | | Acid | | | |
| CTD13:CKP | 13 | Amino | Single | Peptide | KLLNLISKLFCGRDKGPDC |
| | | Acid | | | |
| CTD14:CKP | 14 | Amino | single | Peptide | KLLNLISKLFCGRDKRLYDC |
| | | Acid | | | |
| CTD15:CKP | 15 | Amino | Single | Peptide | CRGDKGPDCKLLNLISKLF |
| | | Acid | | | |
| CTD16:CKP | 16 | Amino | Single | Peptide | KLLNLISKLFCRGDKGPDC |
| | | Acid | | | |
| CTD17:CKP | 17 | Amino | Single | Peptide | KLLNLISKLFCRGDKRLYDC |
| | | Acid | | | |
| CTD18:CKP | 18 | Amino | Single | Peptide | CRGDKGGKLLNLISKLF |
| | | Acid | | | |
| CTD19:CKP | 19 | Amino | single | Peptide | KLLNLISKLFGGCRGDK |
| | | Acid | | | |

### EXAMPLE 2

### Test for Cancer Cell Necrosis Activity of Cell-Killing CKP Fusion Peptide

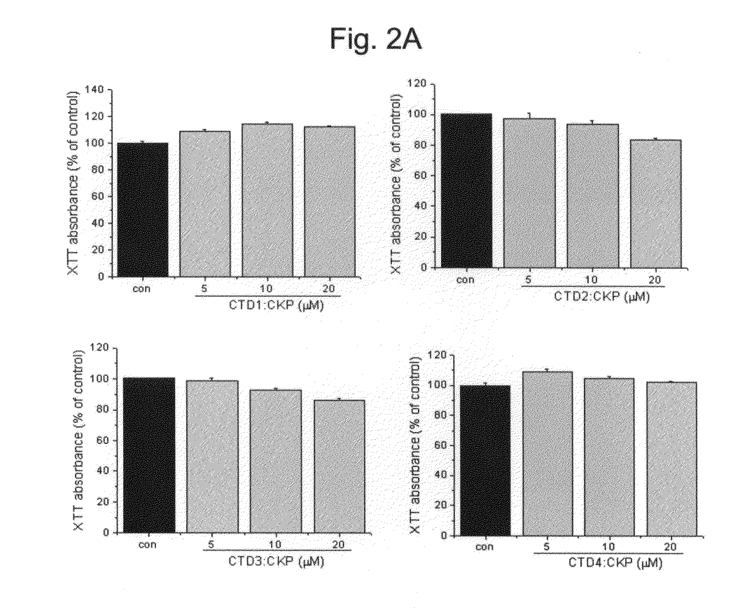

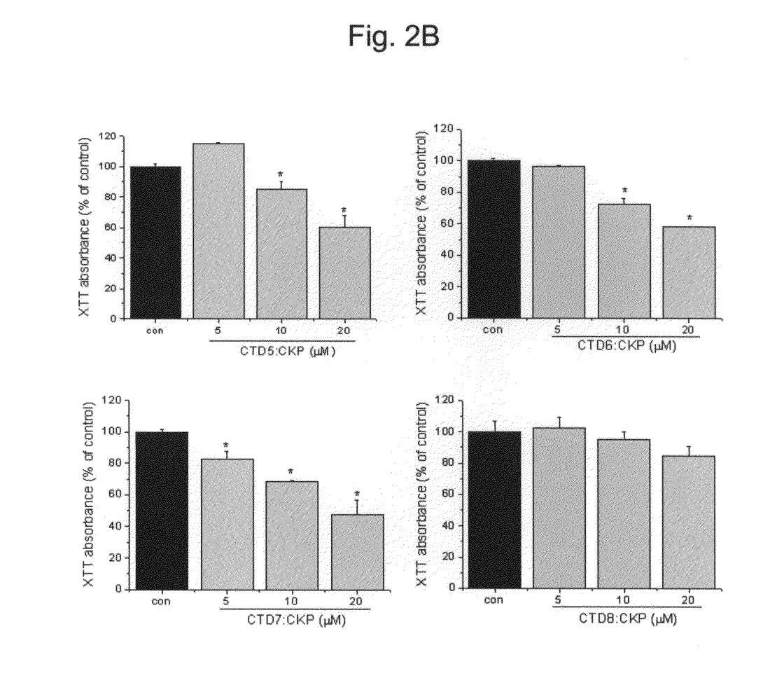

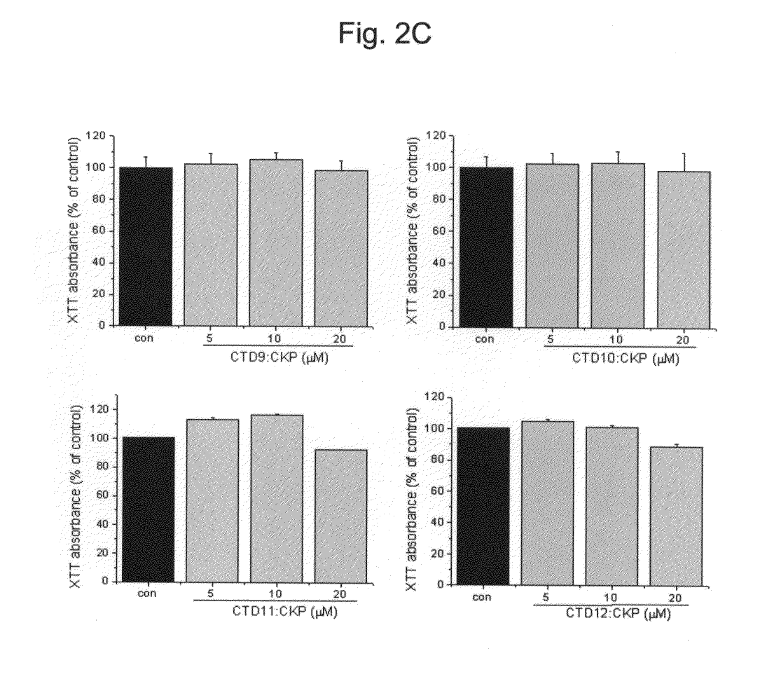

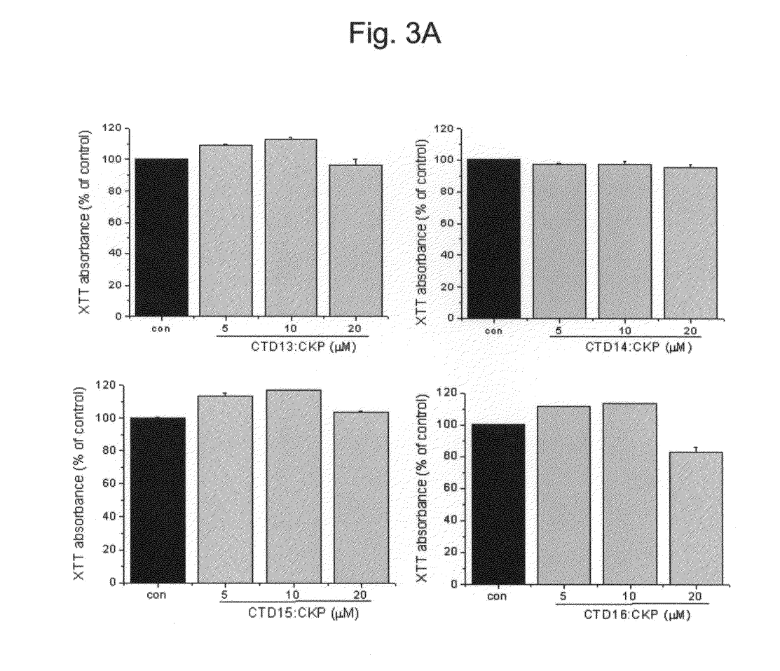

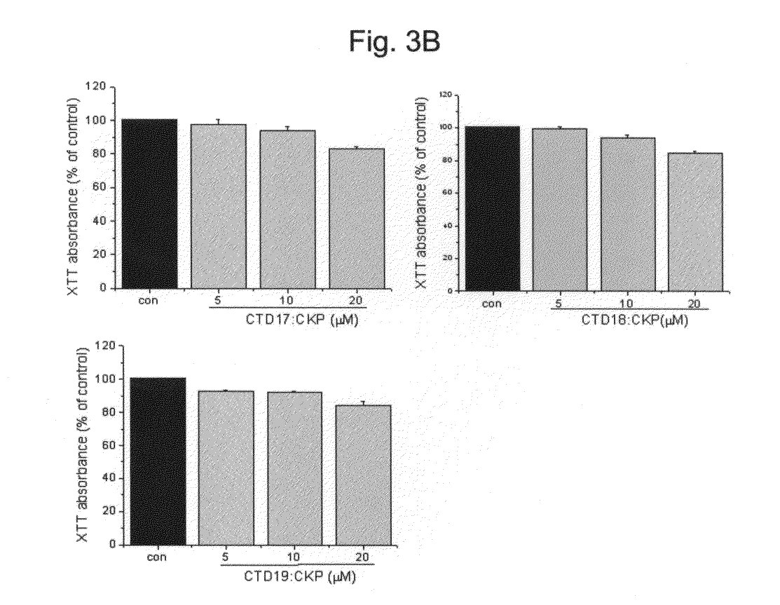

In order to test and select cell necrosis inducing activity of many kinds of cell-killing CKP fusion peptides manufactured in Example 1, CT-26, mouse colon cancer cell line, was cultured, and the culture medium was replaced with culture medium containing 5, 10 and 20 μM of the cell-killing CKP fusion peptides (CTD1-CTD10:CKP) in a 96-well plate, respectively. After 15 min, cell necrosis inducing activity was confirmed by XTT method. As shown in FIGS. 2 to 3, the CTD1:CKP to CTD4:CKP did not show significant cell necrosis inducing activity, and the CTD5:CKP to CTD8:CKP showed remarkably increased cell necrosis inducing activity depending on concentration. Further, the CTD9:CKP to CTD19:CKP did not show significant cell necrosis inducing activity. However, even if the peptides showed excellent cell necrosis inducing activity in the cell necrosis inducing activity test using cancer cell line, those may show toxicity or may not show tumor regression effect in real animal test. Accordingly, tumor regression activity was additionally tested in experimental animals.

### EXAMPLE 3

### Test of Tumor Regression Activity of Cell-Killing CKP Fusion Peptide

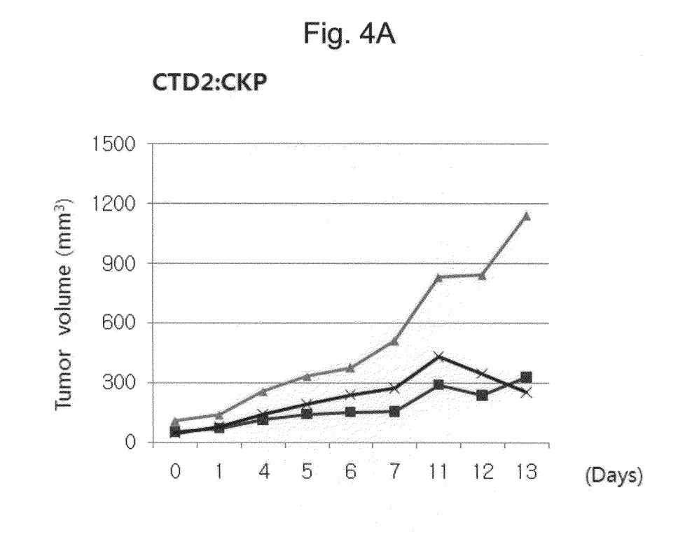

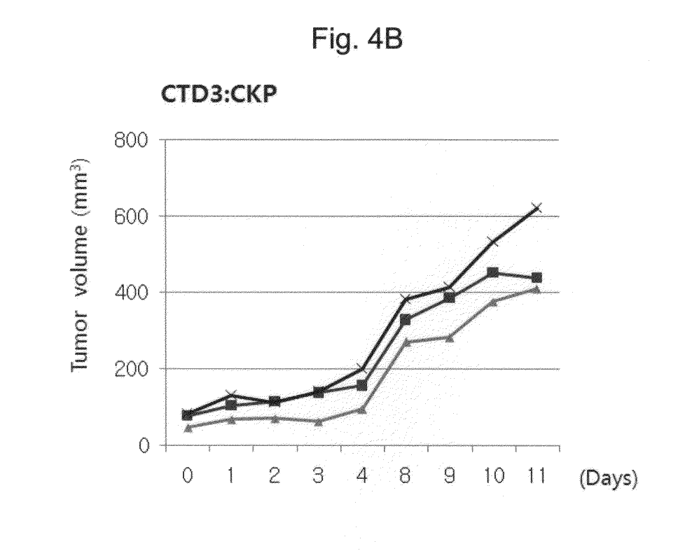

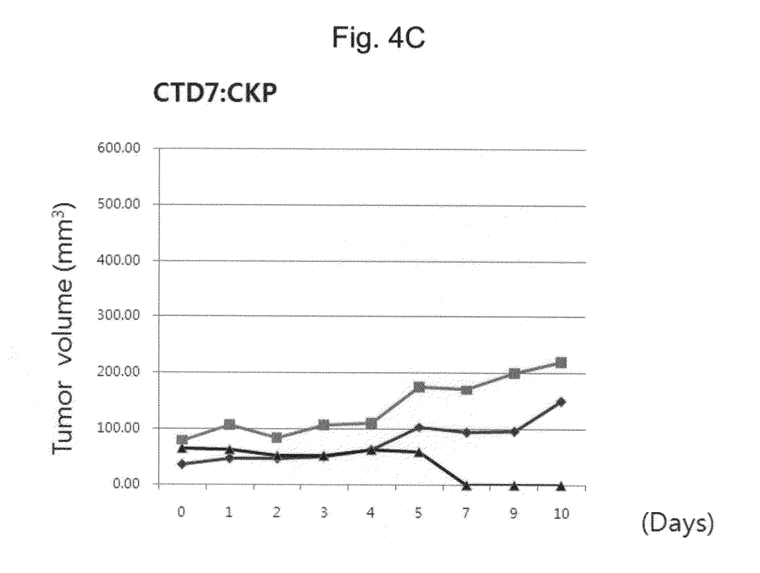

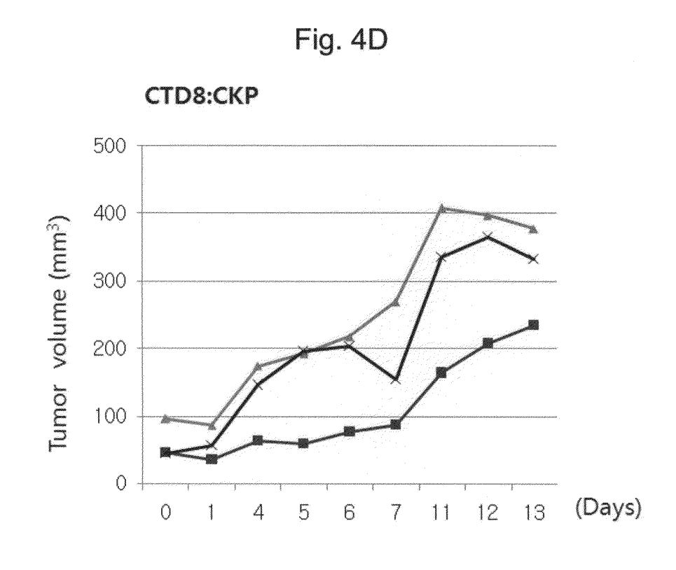

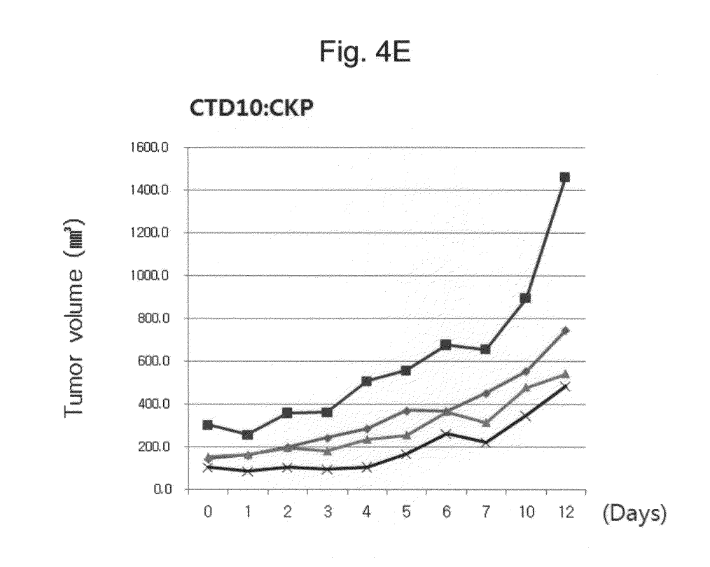

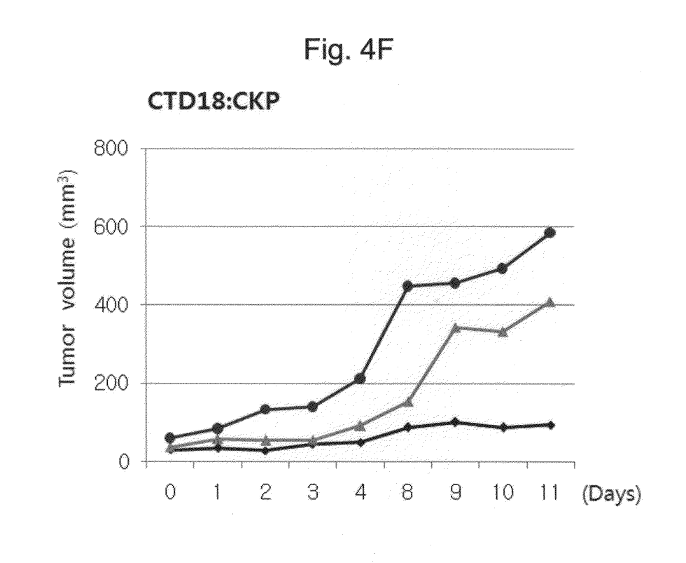

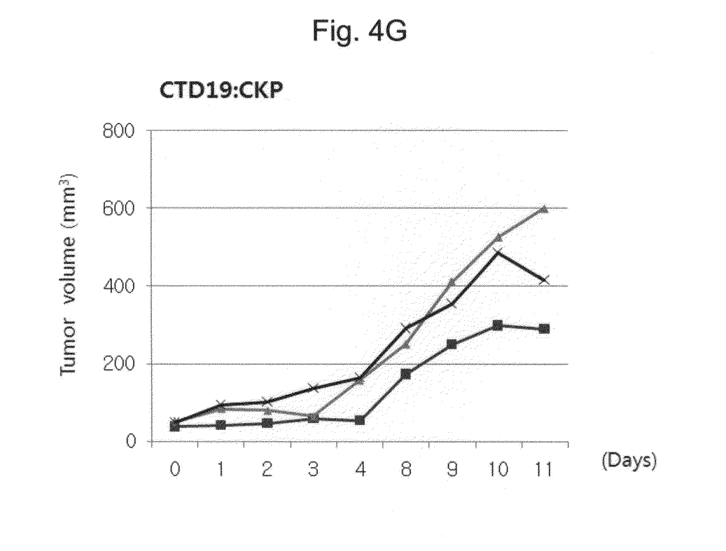

In order to test tumor regression activity of the cell-killing CKP fusion peptides manufactured in Example 1, colon cell line, CT-26 (1.5×10⁵ cells) was subcutaneously injected into dorsal skin of BALB/C mice of 6 weeks old to form tumor. After about 10 days, when the formed tumor volume reach to about 70±10 mm³, the cancer cell specific CKP fusion peptide 100 ul (1 mM) was intravenously injected continuously 2-3 times (1 injection/day) into 3 mice, respectively, and after 10-13 days, change on the tumor tissue volume (length×wide²×0.5) was observed. As a result, tumor regression was not significant in most of the groups injected with the cell-killing CKP fusion peptides; toxicity removing the injected tail by necrosis was found in the groups injected with the CTD4:CKP, CTD5:CKP, CTD6:CKP and CTD17:CKP; and mice of the group injected with the CTDI4:CKP were dead. On the contrary, it was observed that the tumor tissue was completely removed or increase of the tumor tissue volume was remarkably inhibited in the test group intravenously injected with the CTD7:CKP (see FIG. 4). Accordingly, the present invention was completed by selecting the CTD7:CKP, which showed excellent tumor regression activity and no toxicity to the experimental animals as well as excellent effect at the cell necrosis inducing test using a cancer cell line. Table 2 shows in vivo screening summary.

TABLE 2

| Kind of CTD:CKP | In vivo Screening | Movement Activities |
|---|---|---|
| CTD1:CKP | NR | Slow |
| CTD2:CKP | NR | Normal |
| CTD3:CKP | NR | Normal |
| CTD4:CKP | Toxic | Slow |
| CTD5:CKP | Toxic | Slow |
| CTD6:CKP | Toxic | Normal |
| CTD7:CKP | Tumor | Normal |
| | Regression | |
| CTD8:CKP | NR | Slow |
| CTD9:CKP | ND | Not Determined |
| CTD10:CKP | NR | Not Determined |
| CTD11:CKP | ND | Normal |
| CTD12:CKP | ND | Normal |
| CTD13:CKP | ND | Normal |
| CTD14:CKP | Dead | Dead |
| CTD15:CKP | ND | Normal |
| CTD16:CKP | ND | Slow |
| CTD17:CKP | Toxic | Slow |
| CTD18:CKP | NR | Slow |
| CTD19:CKP | NR | Normal |

Movement activity: movement of mouse for 10-30 min after I.V. injection of CTD:CKP 75 ul (1 mM) into BalB/C mouse (about 20 g) was observed.
NR: no regression (no decrease on the tumor volume)
ND: not determined

### EXAMPLE 4

### Test of Human Cancer Cell Death Activity of Cell-Killing CKP Fusion Peptide (CTD7:CKP)

Cell necrosis inducing activity of the cell-killing CKP fusion peptide (CTD7:CKP) selected in Example 3 was tested in human cancer cell lines (HeLa, HCT116, MCF7, A549, BJAB and PC3).

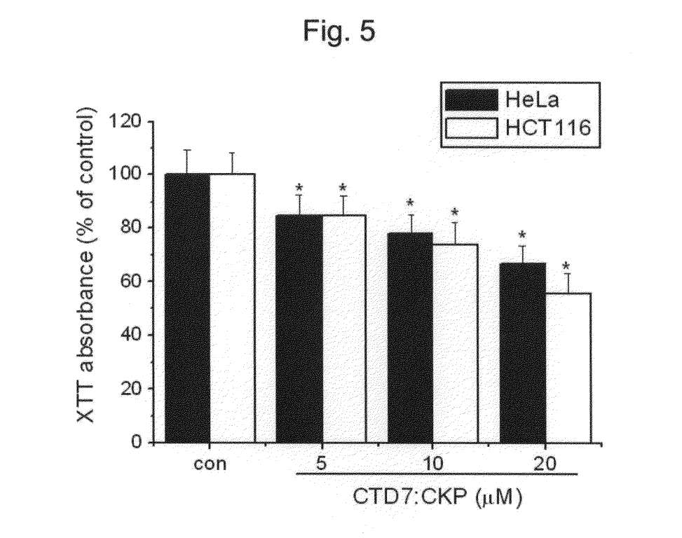

HeLa cells, cervical cancer cell line, and HCT116 cells, human colon cancer cell line, were cultured, respectively, and then the culture medium was replaced with culture medium containing 5, 10 and 20 μM of the cell-killing CKP fusion peptide (CTD7:CKP) in a 96-well plate, respectively. After 15 min, cell necrosis inducing activity was confirmed by XTT method. As shown in FIG. 5, the HeLa cells showed cell necrosis inducing activity of 33% at the treatment concentration of 20 μM, and the HCT116 cells showed cell necrosis inducing activity of 44% at the treatment concentration of 20 μM.

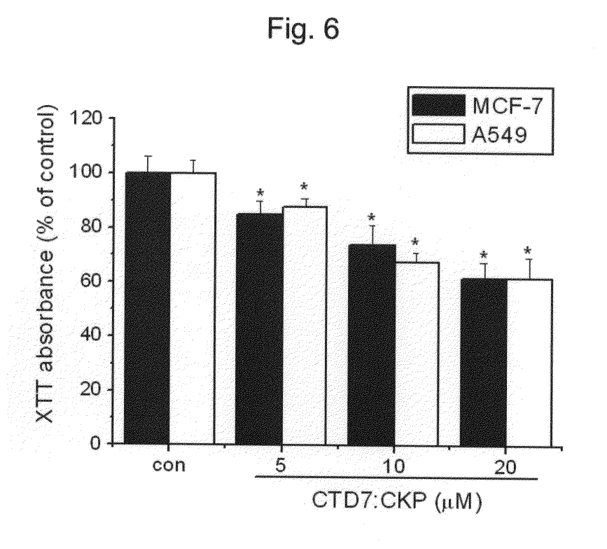

MCF-7 cells, human breast cancer cell line, and A549 cells, human lung cancer cell line, were cultured, respectively, and then the culture medium was replaced with culture medium containing 5, 10 and 20 μM of the cell-killing CKP fusion peptide (CTD7:CKP) in a 96-well plate, respectively. After 15 min, cell necrosis inducing activity was confirmed by XTT method. As shown in FIG. 6, the MCF-7 cells showed cell necrosis inducing activity of 43.2% at the treatment concentration of 20 μM, and the HCT116 cells showed cell necrosis inducing activity of 44% at the treatment concentration of 20 μM.

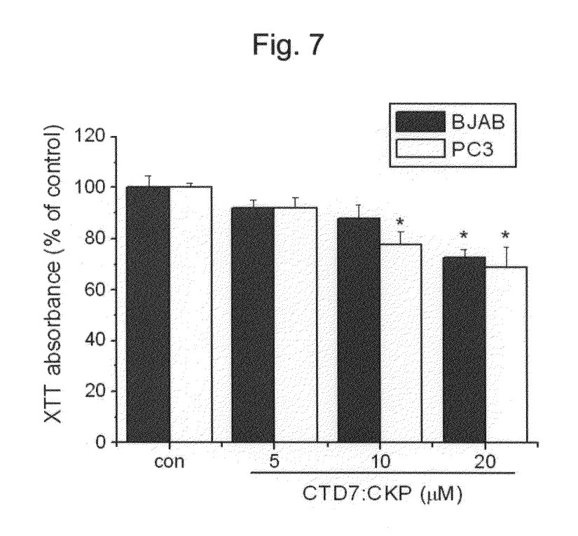

BJAB cells, human B-lymphoma cell line, and PC3 cells, human prostate cancer cell line, were cultured, respectively, and then the culture medium was replaced with culture medium containing 5, 10 and 20 μM of the cell-killing CKP fusion peptide (CTD7:CKP) in a 96-well plate, respectively. After 15 min, cell necrosis inducing activity was confirmed by XTT method. As shown in FIG. 7, the BJAB cells showed cell necrosis inducing activity of 27% at the treatment concentration of 20 μM, and the PC3 cells showed cell necrosis inducing activity of 31% at the treatment concentration of 20 μM.

### EXAMPLE 5

### Test of Normal Cell Necrosis Activity of Cell-Killing CKP Fusion Peptide (CTD7:CKP)

In order to test cell necrosis inducing activity of the cell-killing CKP fusion peptide (CTD7:CKP) selected in Example 3 in normal cells, primary peritoneal macrophages and splenocytes were isolated from BALB/c mice.

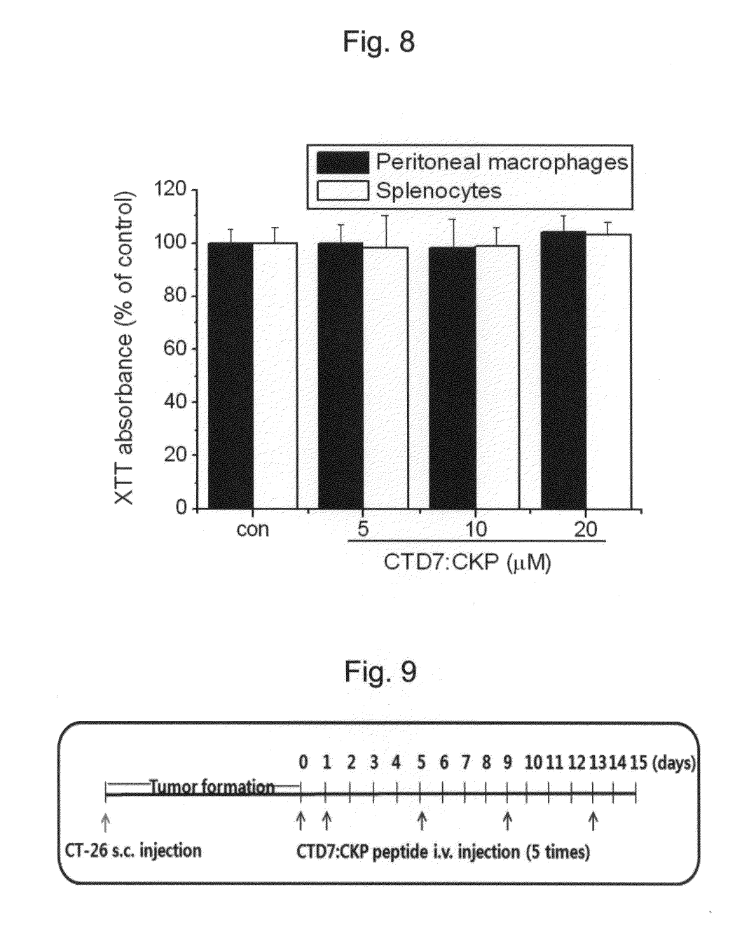

4% (w/v) fluid thioglycollate medium was intraperitoneally injected for 3 days and the cells were collected in RPMI 1640 media and stabilized in a 96-well for 2 hours in a 37° C. 5% CO₂ incubator to obtain the primary peritoneal macrophages. The culture medium was replaced with culture medium containing 5, 10 and 20 μM of the cell-killing CKP fusion peptide (CTD7:CKP), and after 15 min, cell necrosis inducing activity was confirmed by XTT method. As a result shown in FIG. 8, the cell-killing CKP fusion peptide (CTD7:CKP) did not show significant cell-killing activity in the primary peritoneal macrophages as normal cells.

In order to obtain splenocytes, complete RPMI media was put into a cell culture dish, male mouse spleen was isolated, and tissue thereof was crushed and centrifuged. Pellet was washed once with media and centrifuged again, and ACK lysis buffer (0.15 M NH₄Cl, 1 M KHCO₃, 0.01 M Na₂EDTA, pH 7.2-7.4) was added thereto and centrifuged again. Then the pellet was cultured in 5% FBS-containing complete RPMI media for primary culture of the splenocytes, and the media was replaced with culture medium containing 5, 10 and 20 μM of the cell-killing CKP fusion peptide (CTD7:CKP), and after 15 min, cell necrosis inducing activity was confirmed by XTT method. As a result shown in FIG. 8, the cell-killing CKP fusion peptide (CTD7:CKP) did not show significant cell-killing activity in the splenocytes as normal cells.

### EXAMPLE 6

### Test of Tumor Regression Activity of Cell-Killing CKP Fusion Peptide (CTD7:CKP)

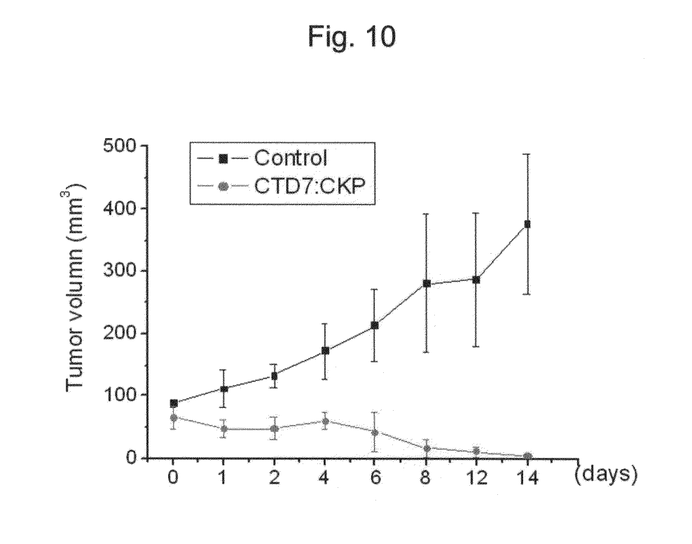

In order to test tumor regression activity of the cell-killing CKP fusion peptide (CTD7:CKP) manufactured in Example 1, as shown in FIG. 9, colon cell line, CT-26 (1.5×10⁵ cells) was subcutaneously injected into dorsal skin of BALB/C mice of 6 weeks old to form tumor. After about 10 days, when the formed tumor volume reach to about 70±10 mm³, the cancer cell specific CKP fusion peptide was intravenously injected 2 times (1 injection/day) and then 3 times (1 injection/4 days) to make the concentration of 210 μg/mouse. After 15 days, change on the tumor tissue volume (length×wide²×0.5) was observed. As a result, the tumor tissue volume was largely decreased in the test group intravenously injected with the cell-killing CKP fusion peptide (CTD7:CKP), but the tumor tissue volume was continuously increased in the control group (FIG. 10).

### EXAMPLE 7

### Test of Hepatocyte Toxicity of Cell-Killing CKP Fusion Peptide (CTD7:CKP)

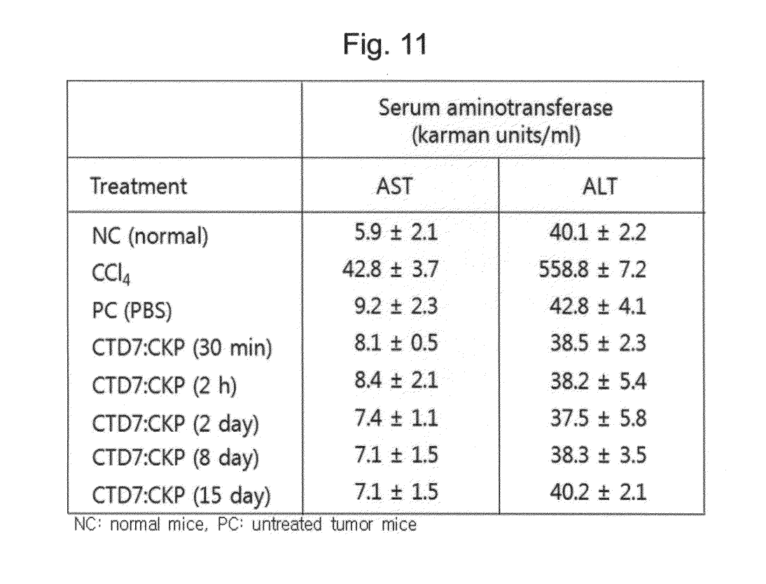

In order to confirm toxicity of the cell-killing CKP fusion peptide (CTD7:CKP), the amounts of alanine amino transferase (ALT) and aspartate amino transferase (AST) released to blood from hepatocytes of the damaged liver were measured and whether the hepatocytes was damaged or not was confirmed, so as to judge toxicity by the cell-killing CKP fusion peptide. As shown in FIG. 11, there was no difference of AST and ALT, as liver damage index, between the blood isolated from the control group not treated with the CTD7:CKP and the blood isolated from the groups treated with the CTD7:CKP by the time.

### EXAMPLE 8

### Test of Histological Change by Cell-Killing CKP Fusion Peptide Depending on Time

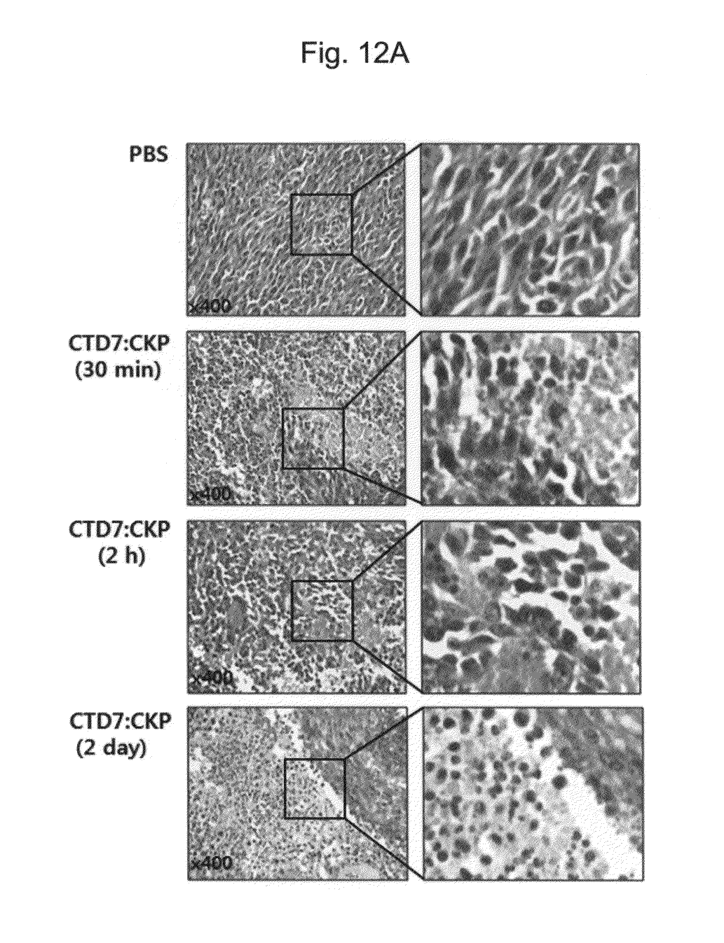

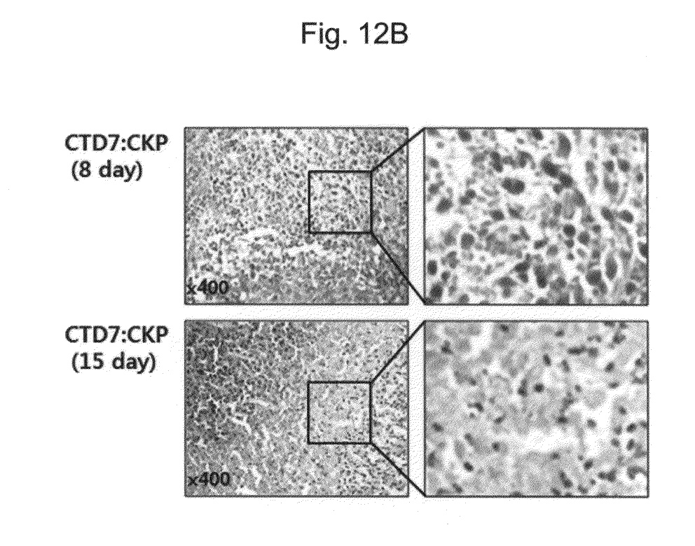

In order to confirm histological change on tumor tissue by the time according to tumor regression activity of the cell-killing CKP fusion peptide (CTD7:CKP), the CTD7:CKP was intravenously injected to the tail vein, and then tumor tissue was isolated from experimental animals after 30 min, 2 hours, 2 days, 8 days and 15 days, respectively. The tissue was put into 4% formaldehyde and fixed at 4° C. After Gloss process, dehydration-clearing-impregnation-embedding process of the tissue was conducted by using a tissue processor (SAKURA tissue tek, VIP-5Jr-J2), and a block was prepared at an embedding center and stored at −20° C. The tissue was serially sectioned (3 μm) by using Microtome (LEICA, RM2135), and a ribbon was floated on a constant-temperature water bath and attached to a slide. The slide was dried, paraffin was removed, and the slide was hydrated. After conducting removal of paraffin-dehydration-Hematoxylin&eosin staining-hydration process, the slide was mounted with non-aqueous mounting media (malinol). The slide was dried in the air, covered with a cover glass, and pathologically observed by using a microscope (Olympusoptical.Co.LTD, V-MD010B) and Magna fire-SP program. As shown in FIG. 12, it was observed that strong cell death was induced in the cancer tissue of the test groups injected with the cell-killing CKP fusion peptide (CTD7:CKP) depending on time, but no cell-killing activity was induced in the cancer tissue of the control group.

### EXAMPLE 9

### Test of Toxicity of Cell-Killing CKP Fusion Peptide in Normal Tissue (Liver)

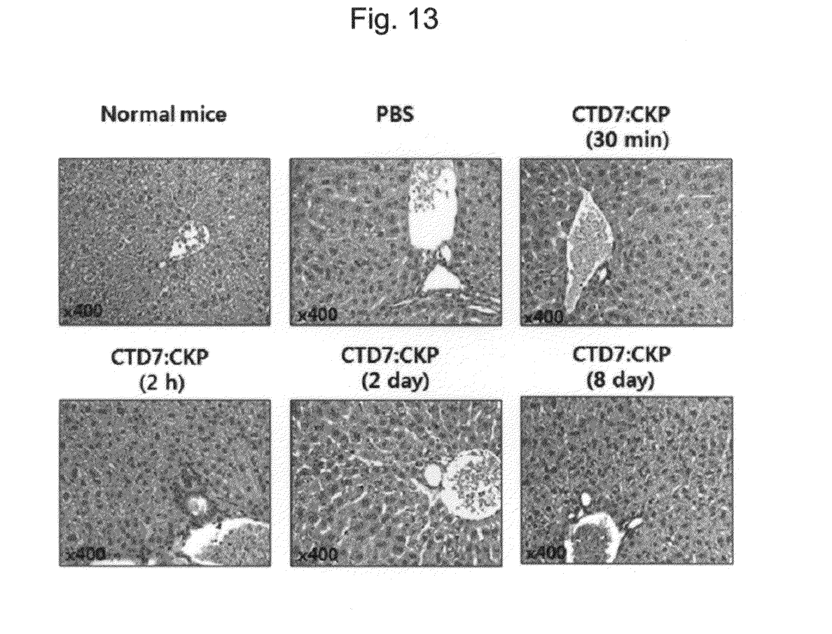

In order to evaluate the degree of toxicity to the liver tissue, liver tissue section was prepared as described in Example 8, and pathologically observed in a normal mice group not treated with the peptide, a tumor-formed group intravenously injected with 0.85% normal saline and groups intravenously injected with the cell-killing CKP fusion peptide by the time. As a result shown in FIG. 13, any significant damage was not observed.

### EXAMPLE 10

### Test of Cell-Killing Activity of CKP Peptide Variant

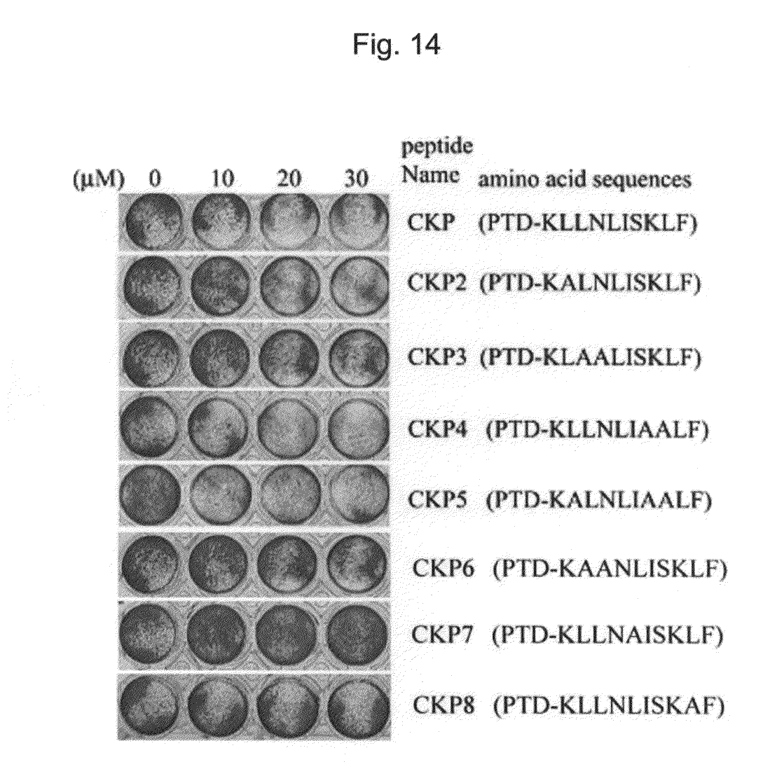

In order to examine whether variants, in which some amino acids of the CKP peptide (SEQ ID NO: 21; KLLNLISKLF) of the present invention are substituted, also have cell-killing activity or not, CKP2 (1 amino acid was substituted) (SEQ ID NO: 22; KALNLISKLF), CKP3 (2 amino acids were substituted) (SEQ ID NO: 23; KLAALISKLF), CKP4 (2 amino acids were substituted) (SEQ ID NO: 24; KLLNLIAALF) and CKP5 (3 amino acids were substituted) (SEQ ID NO: 25; KALNLIAALF), which are CKP peptide variants in which 1-3 of amino acid residues linked to the known protein transduction domain (PTD) having RRRRRRRRG (RQ) sequence are substituted, were synthesized by the procedure described in Example 1. Further, in order to investigate influence of substitution at the leucine's repeat in the CKP, the CKP peptide variants, CKP6 (2^{nd} and 3^{rd} L were substituted to A) (SEQ ID NO: 26; KAANLISKLF), CKP7 (5^{th} L was substituted to A) (SEQ ID NO: 27; KLLNAISKLF) and CKP8 (9^{th} L was substituted to A) (SEQ ID NO: 28; KLLNLISKAF) linked to the RRRRRRRRG (RQ) sequence of the protein transduction domain (PTD) were synthesized by the procedure described in Example 1. In order to test cell-killing activity of the variants, a cervical cancer cell line, HeLa cells were cultured, and the culture medium was replaced with culture medium containing 0, 1, 5, 10, 15, 20, 30 and 40 μM CKP peptide, respectively, followed by further culturing for 24 hours. Living cells attached to the bottom were stained with 100 μl 0.5% crystal violet solution for 10 min, and washed off with distilled water or tap water to remove dead cells floating on the culture medium and also to destain the crystal violet solution. Then, the cells were observed by photography after placing the culture vessel under a fluorescent lamp box as depicted in FIG. 14. The living cells attached to the bottom and stained with the crystal violet were appeared blue color. As shown in FIG. 14, the CKP2, CKP3 and CKP5 peptides showed slightly decreased cell-killing activity, compared with the CKP peptide, but the CKP4 peptide showed better cell-killing activity than the CKP peptide. Further, the CKP6, CKP7, CKP8 peptides, wherein Leucine was substituted, showed much decreased cell-killing activity, compared with the CKP peptide. Accordingly, it was identified that the Leucine sequence is a region playing a critical role in cell-killing activity of the CKP.

### INDUSTRIAL APPLICABILITY

As described above, the cell-killing fusion peptide shows cancer cell necrosis effect and tumor regression effect in a tumor model. Therefore, it can be usefully used for treating various diseases requiring cell death, particularly, as an anti-cancer drug.

1. A cancer targeting cell-killing fusion peptide, which comprises the following cancer cell targeting domain (CTD; R₁-R₇) and cell-killing peptide (CKP), and linker sequence between thereof, and arranged with CTD (R₁-R₇)-Linker-CKP or CKP-Linker-CTD (R₁-R₇) configuration:
(a) the cancer cell targeting domain (CTD; R₁-R₇) consisting of an amino acid sequence expressed as Arg-Xaa-Xaa-Arg-Xaa-Xaa-Arg;
(b) the cell-killing peptide (CKP) consisting of the amino acid sequence of SEQ ID No: 21 (KLLNLISKLF) or its homologous sequences having at least 70% of sequence homology; and
(c) the linker having an amino acid sequence, wherein n=0-5.

2. The cancer targeting cell-killing fusion peptide according to claim 1, wherein the cancer cell targeting domain (CTD; R₁-R₇) is consisting of the amino acid of SEQ ID NO: 20 (RPARPAR).

3. The cancer targeting cell-killing fusion peptide according to claim 1, wherein the cell-killing peptide (CKP) having at least 70% of sequence homology with the amino sequence of SEQ ID NO: 21 is consisting of the amino acid sequence of SEQ ID NO: 22 (KALNLISKLF), SEQ ID NO: 23 (KLAALISKLF), SEQ ID NO: 24 (KLLNLIAALF) or SEQ ID NO: 25 (KALNLIAALF).

4. The cancer targeting cell-killing fusion peptide according to claim 1, wherein in the cell-killing peptide (CKP) having at least 70% of sequence homology with the amino sequence of SEQ ID NO: 21, the 2^{nd}, 3^{rd}, 5^{th} and 9^{th} amino acid sequences of SEQ ID NO: 21 are leucines.

5. The cancer targeting cell-killing fusion peptide according to claim 1, wherein the linker is consisting of amino acid sequence comprising glycine or serine amino acid, wherein n=2-5.

6. A DNA or RNA oligonucleotide encoding the cancer targeting cell-killing fusion peptide according to claim 1.

7. A recombinant vector comprising the DNA oligonucleotide according to claim 6.

7. A cell transformed with the recombinant vector according to claim 7.

9. A method for manufacturing a cancer targeting cell-killing fusion peptide comprising: culturing the transformed cell according to claim 8, and isolating the cancer targeting cell-killing fusion peptide from the cultured cell.

10. A method for manufacturing a cancer targeting cell-killing fusion peptide by chemical synthesis according to solid phase peptide synthesis comprising: sequentially linking amino acids arranged with the CTD (R₁-R₇)-Linker-CKP or CKP-Linker-CTD (R₁-R₇) configuration according to claim 1 to a polymer scaffold, followed by finally separating thereof from the polymer scaffold.

11. An antibody produced by using the cancer targeting cell-killing fusion peptide according to claim 1 as an antigen.

12. A PEG variant of the cancer targeting cell-killing fusion peptide according to claim 1, wherein PEG is linked to the cancer targeting cell-killing fusion peptide.
